(19) United States
(12) Patent Application Publication
(10) Pub. No.: US 2005/0000387 A1
(43) Pub. Date: Jan. 6, 2005

(54) Wood preservative with alkaline copper quaternary

(76) Inventors: Ying Wang, West Chester, PA (US); Shi Hua Zhang, Wilmington, DE (US)

Correspondence Address: E I DU PONT DE NEMOURS AND COMPANY;LEGAL PATENT RECORDS CENTER, BARLEY MILL PLAZA 25/1128, 4417 LANCASTER PIKE, WILMINGTON, DE, 19805, US

(21) Appl. No.: 10/858,103

(22) Filed: Jun. 1, 2004

Related U.S. Application Data
(60) Provisional application No. 60/484,617, filed on Jul. 2, 2003.

(51) Int. Cl. C09D 5/16; C09D 5/18; A61K 31/40; A01N 43/36

(52) U.S. Cl. 106/18.32; 424/638

(57) ABSTRACT

This invention comprises wood preservative compositions based on alkaline copper quaternary formulations having reduced corrosivity to metals by addition of a soluble nitrite salt and a dicarboxylic acid and methods for their use in reducing corrosivity of metals.

### FIELD OF THE INVENTION

This invention relates to the field of wood preservatives based on alkaline copper quaternary (ACQ) formulations.

### BACKGROUND OF THE INVENTION

Wood preservatives are widely used on wood used for fencing, poles, decks, buildings and numerous other applications to protect against rot and wood-attacking insects. Chromate copper arsenate preservatives are effective but have come under increasing regulatory pressure because of ecological and safety problems related to their toxicity. As a result, the U.S. Environmental Protection Agency (EPA) and the wood treatment industry have agreed to phase out chromate copper arsenate treatment from the residential wood market by the end of 2003. As a result, many users are switching to wood preservatives based on alkaline copper quaternary amine (ACQ) formulations. However, the ACQ system is more corrosive to metals than the chromate copper arsenate system. Severe corrosion problems occurred in any treating facilities, including the treatment tank, the preservative solution storage/recovery tank, and the iron piping and valves made of alloy steel. In addition, the treated wood would corrode the fasteners or nails used in any structures, thus reducing the structure life.

European Patent 238413 (1990) discloses a wood preservative composition which provides the desired preservative property using a single vacuum/pressure treatment, reduces corrosion of treatment equipment, and solves the problems of toxicity and copper precipitation. The composition comprises (a) a quaternary ammonium salt having wood preservative properties, (b) a water soluble copper salt, (c) ammonia or a water soluble amine capable of forming a copper complex, and (d) a molybdic acid compound and/or a nitrite, dissolved in an aqueous solvent in a weight ratio of (a)/(b)/(c)/(d) of 1/0.6-1.5 (as copper)/0.05-3.5 (as nitrogen)/0.05-0.8 (as molybdenum or nitrous acid). All tests were based on immersing a metal in a solution.

U.S. Pat. No. 4,622,248 (1986) discloses an aqueous, alkaline wood preservative composition to help prevent deterioration of wood due to fungal decay and insects. The composition comprises copper, specific organic acids including aliphatic dicarboxylic acids containing 2-10 carbon atoms per molecule and their salts, and an ammonia-containing compound sufficient to solubilize the copper and neutralize the acid. There is no teaching of reducing corrosivity.

The American Wood-Preservers' Association (AWPA) is the primary standard-setting body for pressure-treated wood. They test the various proposed wood treating methods, including ACQ-type wood preservatives and others, against a set of rigorous standards. Up to the present time they have approved only three formulations for ACQ wood preservatives. Each of these has 66.7% copper as copper oxide and 33.3% of a quaternary ammonium compound, i.e., a 2 to 1 mixture, dissolved in various solutions. Types B and D use didecyidimethyl ammonium chloride as the quaternary ammonium compound, and Type C uses alkylbenzyldimethyl ammonium chloride. The compositions are dissolved in ethanolamine or ammonia to give aqueous solutions having a pH of from 8.5 tol 1.5. To aid in solubility, the treating solutions also contain carbonate anions. The approved formulations are rated satisfactory in terms of wood preservation but all cause some corrosion to metals.

There is a need for an ACQ wood preservative formulation having reduced corrosivity both to the metals in the treatment system, such as equipment and piping, and to the metal fasteners used in the treated wood in various structural applications. The present invention provides such a wood preservative composition and methods for reducing corrosion.

### SUMMARY OF THE INVENTION

The present invention comprises an improved aqueous alkaline composition for wood preservation containing a copper compound and a quaternary amine salt, wherein the improvement comprises reducing corrosivity to metals by addition of from about 0.01 to about 1.0% by weight of a soluble nitrite salt expressed as sodium nitrite and from about 0.05 to about 1.0% by weight of a dicarboxylic acid.

The present invention further comprises an aqueous alkaline composition for wood preservation comprising a copper compound, didecyidimethyl ammonium chloride or alkylbenzyldimethyl ammonium chloride or both, from about 0.05% to about 0.8% of a soluble nitrite salt expressed as sodium nitrite and from about 0.05% to about 0.8% of a dicarboxylic acid mixture, said mixture comprising from about 32% to about 45% dodecanedioic acid, from about 32% to about 45% undecanedioic acid, from about 6% to about 9% sebacic acid, from about 2.5% to about 4.0% azelaic acid, from about 2.0% to about 3.2% suberic acid, from about 1.6% to about 2.6% pimelic acid and from about 4.0% to about 12.0% of other dicarboxylic acids, wherein all percentages are by weight.

The present invention further comprises a method for reducing corrosivity to metal of a wood preservative composition containing a copper compound and a quaternary compound comprising adding to the composition at least 0.01% of a soluble nitrite salt expressed as sodium nitrite and at least 0.05% of a dicarboxylic acid.

The present invention further comprises a method for reducing corrosion of metal equipment used in wood treatment processes comprising treating the wood with a preservative composition comprising a copper compound, a quaternary ammonium compound, a soluble nitrite salt and a dicarboxylic acid.

### DETAILED DESCRIPTION

Trademarks are shown herein by capitalization.

“ACQ” is used herein to mean alkaline copper quaternary amine composition.

The composition of the present invention comprises a copper compound, a quaternary amine salt, a nitrite salt and a dicarboxylic acid. The addition of the nitrite salt and dicarboxylic acid provide improved anti-corrosion properties for metal over known ACQ-type wood preservative compositions. Preferably the composition of this invention comprises a copper compound and an alkyl or aralkyl quaternary ammonium chloride in a weight ratio of copper, expressed as copper oxide, to the quaternary salt of about 2 to 1, dissolved in an amine or ammonia solution, wherein the improvement is that it also contains at least 0.01% by weight of sodium nitrite and at least 0.05% by weight of a dicarboxylic acid relative to the total weight of the formulation. Preferably the alkyl or aralkyl quaternary ammonium compound is selected from the group consisting of didecyldimethyl ammonium chloride (DDAC) or alkylbenzyldimethyl ammonium chloride (BAC) or both, and the dicarboxylic acid has at least 10 carbon atoms per molecule.

The compositions of the present invention are prepared using methods for making AWPA preservatives Types B, C and D with addition of the nitrite and dicarboxylic acid. Procedures for making AWPA Preservative Types B, C and D are well known. Typically, copper oxide is dissolved in ethanolamine and/or ammonia and a carbonate to give an aqueous solution, to which is added a quaternary amine salt having wood preservation properties. Currently, either didecyldimethyl ammonium chloride (DDAC) or alkylbenzyldimethyl ammonium chloride (BAC) or both is used as the quaternary salt. The proportions are such that the final solution has a ratio of copper oxide to quaternary ammonium salt of about 2 to 1, wherein the copper oxide is expressed as CuO and the quaternary ammonium compound is expressed as DDAC or BAC. The end result is an aqueous solutions having a pH of from about 8.5 tol 1.5 and containing about 66.7% copper oxide and 33.3% of the quaternary ammonium compound, dissolved in ethanolamine or ammonia and a soluble carbonate. The nitrite salt and dicarboxylic acid are then added.

The copper salt used in the compositions and methods of the present invention is any inorganic or organic copper salt, such as copper oxide, copper sulfate, copper acetate, basic copper carbonate, copper bicarbonate, ammoniacal copper complex, or combinations of two or more thereof. Copper oxide is preferred. To solubilize the copper oxide, any nitrogen compound that reacts with copper to form a water-soluble complex may be used, in an amount required to promote solubility of the copper salt. Preferred compounds are ammonia and ethanolamine because of availability and ease of handling. Optionally a soluble carbonate anion may be added in an amount to promote ready solubility of the copper oxide. The soluble carbonate anion may be provided by adding carbon dioxide, carbonic acid, sodium carbonate, ammonium bicarbonate or any other soluble carbonate or bicarbonate salt.

The quaternary amine salt used in the compositions and methods of the present invention having wood preservation properties are selected from any known in the art. Preferably it is selected from amine salts having both long-chain alkyl or benzyl groups and short-chain alkyl groups, wherein by long-chain alkyl groups is meant those containing from 8 to 18 carbons, and by short-chain alkyl groups is meant those containing from 1 to 3 carbons. The quaternary amine salt may have either one or two long-chain alkyl or benzyl groups with the remainder short-chain alkyl groups. As examples, didecyldimethyl ammonium chloride, dioctyl dimethylammonium chloride, dilauryldimethylammonium chloride, distearyldimethylammonium chloride, and alkylbenzyldimethylammonium chloride are mentioned. Didecyldimethyl ammonium chloride and alkylbenzyldimethylammonium chloride are preferred because of their acceptance by the trade.

The inventive composition comprises the above ACQ compositions, to which sufficient amounts of (1) a soluble nitrite salt and (2) a dicarboxylic acid are added to reduce the corrosivity of the ACQ formulation to metals. Preferably the ACQ formulation used in the compositions of the present invention comprises the formulations of AWPA Type B, C or D. Most preferably it comprises the formulation of AWPA Type C and D.

The soluble nitrite salt is selected from the group composed of alkali metals such as sodium nitrite, potassium nitrite, lithium nitrite, ammonium nitrite, or combinations of two or more thereof. Sodium nitrite is preferred because of its ready availability. The amount of nitrite is from about 0.01% to about 1.0% by weight expressed as sodium nitrite relative to the weight of total formulation. Preferably the amount of nitrite is from about 0.05 to about 0.8%, more preferably from about 0.05 to about 0.5%, and still more preferably from about 0.08 to about 0.2%. Amounts lower than 0.01% are insufficient to prevent corrosion. Amounts higher than about 1.0% may also be used, but add unnecessary weight and treatment cost to the product.

Dicarboxylic acids are all solids. The lower members are appreciably soluble in water and most of them dissolve easily in lower alcohols. Borderline solubility in water is found in dicarboxylic acids having 6 to 7 carbon atoms per molecule with a solubility of 2-3 g/100 g of water. Any of the known dicarboxylic acids which can be dissolved into solution are useful in the compositions and methods of the present invention. Preferred are dicarboxylic acids having at least 10 carbon atoms per molecule. For retaining in treated wood permanently, dicarboxylic acids having 10 to 20 carbon atoms per molecule are selected. Preferred are dicarboxylic acids having 10 to 16 carbon atoms.

A mixture of such acids is useful in the present invention. The dicarboxylic acids having from 10 to 16 carbon atoms per molecule are preferred for use in the mixture. The mixture may also contain smaller amounts of dibasic acids of other chain lengths. Preferred is a dibasic acid mixture containing more than about 70% by weight dibasic acids with 10 to 12 carbon atoms per molecule. Most preferred is a dibasic acid mixture containing about 32% to 45% by of dodecanedioic acid, 32% to 45% undecanedioic acid, 6% to 9% sebacic acid, 2.5% to 4.0% azelaic acid. 2.0% to 3.2% suberic acid, 1.6% to 2.6% pimelic acid and 4.0% to 12.0% of other dibasic acids, wherein all percentages are by weight. This mixture is available from the E. I. du Pont de Nemours and Company, Wilmington, Del. under the name of CORFREE M1 dibasic acids.

The amount of dicarboxylic acids used in the composition of the present invention is from about 0.05% to about 1.0% by weight relative to the weight of total formulation to provide effective reduction of corrosion. Preferably the amount of carboxylic acids is from about 0.05% to about 0.8%, more preferably from about 0.08% to about 0.8%, and still more preferably from about 0. 1% to about 0.4%, each by weight of the composition. Amounts lower than 0.05% may be insufficient to prevent corrosion. Amounts higher than 1.0% may also be used but increase the total weight and treatment cost for the product.

Other ingredients may be added to the compositions of the present invention to improve biocidal or fungicidal effectiveness, prevent mildew, improve stability, or for other purposes. See for example U.S. Pat. No. 5,853,766 herein incorporated by reference.

The present invention also provides a method to obtain reduced metal corrosivity in a wood preservative composition containing copper and a quaternary compound comprising adding a soluble nitrite salt and a dicarboxylic acid. This method includes not only those ACQ compositions already approved by the AWPA, but also any ACQ wood preservative. By this is meant any aqueous alkaline composition containing a copper compound and a quaternary amine salt having wood preservation properties. The inventive method is the addition to the ACQ wood preservative composition of 0.01 % to 1.0% by weight of a soluble nitrite salt expressed as sodium nitrite and 0.05% to 1.0% by weight of a dicarboxylic acid having from 10 to 20 carbon atoms per molecule wherein the percentages are expressed relative to the total weight of the formulation, and wherein that the composition has reduced corrosivity to metals.

The present invention further provides a method for reducing corrosion of metal equipment used to treat wood with wood preservative compositions comprising use of the above-described wood preservative compositions of this invention. The treating solution is applied to wood by dipping, soaking, spraying, brushing, or any other well-known means. Vacuum and/or pressure techniques are also used to impregnate the wood with the composition of the present invention. Prior to treatment the wood is first seasoned to remove free water to enhance absorption. Often it is desirable to carry out cutting, machining and boring of the wood prior to treatment to avoid later exposing untreated surfaces which then require further treatment. Use of the composition of the present invention in the treatment process results in decreased corrosion of metal treatment equipment. The composition can also potentially be used to reduce corrosion of metal in wood structural applications such as fasteners, nails, screws, bolts, and similar construction elements, compared to treatment with ACQ wood preservative compositions.

### Test Methods

To test corrosion in the treating system equipment AWRA test E17-99 was employed in the examples hereinafter except that metal fasteners were employed instead of metal coupon. We chose carbon steel fasteners for the test to represent the material used in most treatment equipment. Details of this method are available at American Wood-Preservers' Association, Standards 2001, E17-99, page 455, herein incorporated by reference. Because equipment used to treat wood with a preservative composition is in direct contact with that composition, the test uses the technique of immersing metal into the solution to be tested. We also tested the corrosion rate of hot-galvanized fasteners by the above-mentioned method as an initial screen for the fastener in treated wood.

### EXAMPLES

### Examples 1-6

First, 1000 g of “COPPER-COUNT-N” solution was charged into a 2000 mL three neck-round bottom flask equipped with a mechanical stirrer and a thermometer. “COPPER-COUNT-N” is a copper ammonium carbonate solution containing 8% copper (as metal), available from Mineral Research Development in Harrisburg, N.C. Then 280.2 g of ethanolamine was added, portion-by-portion, to the flask under stirring while controlling the temperature so as not to exceed 45° C. After stirring 30 minutes, 63.7 g of 80% solution of didecyldimethyl ammonium chloride (DDAC) was added to the mixture and stirred vigorously for another 30 minutes. This provided a concentrate of a Type D formulation of an ACQ wood preservative.

A 1 gallon solution of 2% of the ACQ solution of Example 1 was made by dilution of the concentrated solution (666.37 g, 11.37%) with 3118.63 g of deionized water. The potential corrosion inhibitors CORFREE M1 and sodium nitrite, in amounts as listed in Table 1 were then added to a 2% solution and shaken or stirred until dissolved. CORFREE M1 is a dibasic acid mixture containing 32 to 45% by of dodecanedioic acid, 32 to 45% undecanedioic acid, 6 to 9% sebacic acid, 2.5 to 4.0% azelaic acid. 2.0 to 3.2% suberic acid, 1.6 to 2.6% pimelic acid and 4.0 tol 2.0% of other dibasic acids, where all percentages are by weight, available from the E. I. du Pont de Nemours and Company, Wilmington, Del. Deionized water and the 2% ACQ solution were tested as controls.

Carbon steel fasteners (purchased from Lowe's, Wilmington, Del., as “THS Fluted Masonry, GripRite Fas'ners”) were cleaned with fine garnet sandpaper, washed with a solution of an alcohol/acetone mixture, then wiped dry and weighed to obtain the initial weight. Galvanized fasteners (purchased from Lowe's, Wilmington, Del., as “Primeguard” phillips-head exterior screws) were cleaned with water and a solution of an alcohol/acetone mixture, then wiped dry and weighed to obtain the initial weight. The solutions and fasteners prepared as above were then tested for corrosion by immersing the fasteners in a 20 mL vial containing 10 ml of solution, with the cap covered loosely to minimize solution evaporation and ensure a free flow of air. The fasteners were left immersed for 10 days at room temperature. The fasteners were removed from the solution, cleaned to remove rust, and weighed. The calculations were based on the following equations and results are shown in Table 1.

$$\text{Weight loss \%} = 100 \times \frac{\text{(initial weight} - \text{after exposure weight)}}{\text{initial weight}}$$

$$\text{Inhibitive Efficiency \%} = 100 \times \frac{( standard ( ACQ ) weight loss - weight loss )}{\text{standard weight loss}}$$

TABLE 1

| | | | Corrosion rate, | | | |
|---|---|---|---|---|---|---|
| | | | 10 days weight loss % | | Inhibitive efficiency % | |
| | Sodium | CORFREE | Carbon | Hot-dip | Carbon | Hot-dip |
| Ex. | nitrite, ppm | M1, ppm | Steel | Galvanized | Steel | Galvanized |
| 1 | 450 | 2100^{a} | 0.00265 | 0.3522 | 95.81 | 60.95 |
| 2 | 750 | 1500^{a} | −0.0103 | 0.1706 | 100 | 81.08 |
| 3 | 900 | 1200^{a} | −0.00528 | 0.0073 | 100 | 99.19 |
| 4 | 450 | 2100^{b} | 0.0 | 0.1285 | 100 | 85.75 |
| 5 | 750 | 1500^{b} | 0.0316 | 0.05549 | 50.08 | 93.85 |
| 6 | 900 | 1200^{b} | −0.0077 | 0.1287 | 100 | 85.73 |
| DI | 0 | 0 | 0.1661 | 0 | | |
| Con | 0 | 0 | 0.0633 | 0.9020 | 0 | 0 |

Note:
Test solutions were 2% ACQ-D solution plus two corrosion inhibitors, sodium nitrite and CORFREE M1.
Con = control = 2% ACQ-D solution.
DI = deionized water.
ppm is micrograms per gram.
^{a}CORFREE M1 30% solution in methylethylamine:triethylamine (1:1),
^{b}Solid CORFREE M1

The addition of sodium nitrite and dibasic acids to the ACQ-D solution provided a wood preervative composition which effectively inhibited the corrosion of both mild steel and galvanized fasteners with inhibitive efficiency of 100% and 99% in 10 days respectively. The corrosion inhibitive efficiency for galvanized material depended on the ratio of two additive components.

### Examples 7-9

Futher tests were carried out according to the process of Example 1 for corrosion inhibition on galvanized screws. The results are shown in Table 2. These results indicated that the combination of sodium nitrite and CORFREE M1 was more effective than either one used separately.

TABLE 2

| | | 2 day Wt. | 6 day Wt. | 12 day Wt. |
|---|---|---|---|---|
| Ex. | Solution* | Loss % | Loss % | Loss % |
| DI | Deionized Water | −0.0074 | 0 | 0 |
| Con | 2% ACQ-D | 0.0936 | 0.4642 | 1.029 |
| 7 | 900 ppm NaNO2 | 0.0655 | 0.0728 | 0.1128 |
| 8 | 1200 ppm CORFREE M1 | 0.0886 | 0.2252 | 0.4025 |
| 9 | 900 ppm NaNO2 + | 0.0448 | 0.0112 | −0.1675** |
| | 1200 ppm COREREE M1 | | | |

Note:
*testing solutions were in 2% ACQ-D solution.
**Negative wt. loss % was due to un-cleaned surface but it was very light corrosion.
ppm is micrograms per gram.

### Comparative Examples A-D

The process of Example 1 was repeated using a combination of CORFEE M1 and boric acid in the amounts listed in Table 3. Weight loss was measured after 2, 6 and 12 days of immersion of the hot-dipped galvanized screws in the solution. The mixture of boric acid and CORFREE M1 did not show any synergistic effect for inhibition of corrosion.

TABLE 3

| | | 2 | 6 | |
|---|---|---|---|---|
| | | Day Wt | Day Wt | 12 Day wt. |
| | Solution* | Loss % | Loss % | Loss % |
| Comp A | 2000 ppm COREREE M1 | 0.0834 | 0.2211 | 0.5800 |
| Comp B | 2000 ppm Boric acid | 0.0879 | 0.4357 | 1.0252 |
| Comp C | 2000 ppm CORFREE M1 + | 0.0964 | 1.0044 | 1.1156 |
| | Boric acid (1:1) | | | |
| Comp D | 4000 ppm COREREE M1 + | 0.1033 | 0.7268 | 0.9962 |
| | boric acid (1:1) | | | |

Note:
*testing solutions are in 2% ACQ-D solution.
ppm is micrograms per gram.

1. An improved aqueous alkaline composition for wood preservation containing a copper compound and a quaternary ammonium salt, wherein the improvement comprises reducing corrosivity to metal by addition of from about 0.01% to about 1.0% by weight of a soluble nitrite salt expressed as sodium nitrite and from about 0.05% to about 1.0% by weight of a dicarboxylic acid.

2. The composition of claim 1 wherein the quaternary ammonium salt is selected from the group consisting of didecyldimethyl ammonium chloride, alkylbenzyldimethyl ammonium chloride, and combinations thereof.

3. The composition of claim 2 wherein the copper compound is selected from the group consisting of copper oxide, copper sulfate, copper citrate, basic copper carbonate, copper bicarbonate, ammoniacal copper complex, and combinations thereof.

4. The composition of claim 1 wherein the amount of a soluble nitrite salt is from about 0.05% to about 0.8% by weight and the amount of dicarboxylic acid is from about 0.05% to about 0.8% by weight.

5. The composition of claim 3 wherein the amount of a soluble nitrite salt is from about 0.05 to about 0.5% by weight, and the amount of dicarboxylic acid is from about 0.08 to about 0.8% by weight.

6. The composition of claim 4 wherein the amount of a soluble nitrite salt is from about 0.08 to about 0.2% by weight and the amount of dicarboxylic acid is from about 0.1 to about 0.4% by weight.

7. The composition of claim 1 wherein the dicarboxylic acid has at least ten carbon atoms per molecule.

8. The composition of claim 7 wherein the dicarboxylic acid is a mixture of acids having about 10 to about 16 carbon atoms per molecule.

9. The composition of claim 8 wherein the dicarboxylic acid mixture contains from about 32% to about 45% dodecanedioic acid, from about 32% to about 45% undecanedioic acid, from about 6% to about 9% sebacic acid, from about 2.5% to about 4.0% azelaic acid, from about 2.0% to about 3.2% suberic acid, from about 1.6% to about 2.6% pimelic acid and from about 4.0% to about 12.0% of other dicarboxylic acids, wherein all percentages are by weight.

10. The composition of claim 1 wherein the corrosivity is measured by a method wherein said metal is immersed in a solution.

11. An aqueous alkaline composition for wood preservation comprising a copper compound, didecyidimethyl ammonium chloride or alkylbenzyldimethyl ammonium chloride, from about 0.05% to about 0.8% of a soluble nitrite salt expressed as sodium nitrite and from about 0.05% to about 0.8% of a dicarboxylic acid mixture, said mixture comprising from about 32% to about 45% dodecanedioic acid, from about 32% to about 45% undecanedioic acid, from about 6% to about 9% sebacic acid, from about 2.5% to about 4.0% azelaic acid, from about 2.0% to about 3.2% suberic acid, from about 1.6% to about 2.6% pimelic acid and from about 4.0% to about 12.0% of other dicarboxylic acids, wherein all percentages are by weight.

12. A method for reducing corrosivity to metal of a wood preservative composition containing a copper compound and a quaternary ammonium compound comprising adding to the composition at least 0.01% of a soluble nitrite salt and at least 0.05% of a dicarboxylic acid.

13. The method of claim 12 wherein the amount of a soluble nitrite salt is from about 0.01% to about 1.0% by weight and the amount of dicarboxylic acid is from about 0.05 to about 1.0% by weight.

14. The method of claim 13 wherein the dicarboxylic acid is a mixture of acids having about 10 to 16 carbon atoms per molecule.

15. The method of claim 14 wherein the dicarboxylic acid mixture contains from about 32% to about 45% dodecanedioic acid, from about 32% to about 45% undecanedioic acid, from about 6% to about 9% sebacic acid, from about 2.5% to about 4.0% azelaic acid, from about 2.0% to about 3.2% suberic acid, from about 1.6% to about 2.6% pimelic acid and from about 4.0% to about 12.0% of other dicarboxylic acids, wherein all percentages are by weight.

16. A method for reducing corrosion of metal equipment used in wood treatment processes comprising treating the wood with a preservative composition comprising a copper compound, a quaternary ammonium compound, a soluble nitrite salt and a dicarboxylic acid.

17. The method of claim 16 wherein the amount of a soluble nitrite salt is from about 0.01% to about 1.0% by weight and the amount of dicarboxylic acid is from about 0.05% to about 1.0% by weight.

18. The method of claim 17 wherein the dicarboxylic acid is a mixture of acids having about 10 to 16 carbon atoms per molecule.

19. The method of claim 18 wherein the dicarboxylic acid mixture contains from about 32% to about 45% dodecanedioic acid, from about 32% to about 45% undecanedioic acid, from about 6% to about 9% sebacic acid, from about 2.5% to about 4.0% azelaic acid, from about 2.0% to about 3.2% suberic acid, from about 1.6% to about 2.6% pimelic acid and from about 4.0% to about 12.0% of other dicarboxylic acids, wherein all percentages are by weight.
